(19) United States
(12) Patent Application Publication
(10) Pub. No.: US 2025/0325221 A1
(43) Pub. Date: Oct. 23, 2025

(54) CERVIX STIFFNESS MEASUREMENT SYSTEM AND METHOD

(71) Applicants: Emily Pogue, New York, NY (US); Joyce Liu, San Francisco, CA (US); Hannah Ballard, New York, NY (US); Aala Nasir, Los Angeles, CA (US); Kiarra Lavache, New York, NY (US)

(72) Inventors: Emily Pogue, New York, NY (US); Joyce Liu, San Francisco, CA (US); Hannah Ballard, New York, NY (US); Aala Nasir, Los Angeles, CA (US); Kiarra Lavache, New York, NY (US)

(21) Appl. No.: 18/638,047

(22) Filed: Apr. 17, 2024

(51) Int. Cl. A61B 5/00

(52) U.S. Cl. CPC A61B 5/4331; A61B 5/0053; A61B 5/6875; A61B 5/7275; A61B 5/742

(57) ABSTRACT

A system and method for determining cervical stiffness in a patient, to help identify the risk of preterm birth in pregnant patients, based on abnormal changes in cervical stiffness during pregnancy. A probe is inserted to engage the patient's cervix and activated to deform the cervix a predetermined amount. The amount of force needed for the probe to deform the cervix the predetermined amount is measured and a cervical stiffness value can be displayed, based on how much force was needed to deform the cervix the predetermined amount. The probe can include a piston, cuff or loop or an expanding head to deform the cervix. The probe can include the related electronics to activate the probe and display the results as well as preterm birth risk warnings.

### CROSS REFERENCE TO RELATED APPLICATIONS

This application claims the benefit of and priority to provisional application Ser. No. 63/460,366, filed Apr. 19, 2023, the contents of which are incorporated herein by reference.

### BACKGROUND OF THE INVENTION

The invention relates generally to a new and useful system and method for measuring cervix stiffness to evaluate the risk of preterm birth in pregnant patients.

In the United States, 10% of children are born preterm, which is a significant contributor to infant mortality. Currently, cervical shortening is commonly used to establish a risk of preterm birth. Patients who are considered at high risk for preterm birth due to risk factors such as age or previous preterm births are often recommended to have transvaginal ultrasounds to measure cervical length and detect premature shortening. However, patients who are considered low risk are typically not recommended to have a transvaginal ultrasound, despite the fact that many who deliver preterm have no known risk factors. Thus, the current commonly used procedures have not been fully satisfactory for predicting the risk of a preterm birth.

FIG. 1A shows the relationship of cervical length and gestational age in weeks. See, Parra-Saavedra, M., “Prediction of preterm birth using the cervical consistency index”, Ultrasound in Obstetrics & Gynecology, April 2011, the contents of which are incorporated herein by reference. Although cervical length measurements are often used to assess preterm birth risk, it can be seen that there is an unsatisfactory correlation between cervical length and preterm birth. Accordingly, cervical length can be considered an ineffective predictor of preterm birth risk. Thus, FIG. 1A shows that current systems and methods for measuring cervical length have not been adequate to predict preterm birth.

It can be seen that since cervical length measurements have been determined to be an unsatisfactory indicator for preterm birth risk, there is an unfulfilled need for accessible and proper monitoring of pregnant patients to identify preterm birth risk factors over the course of all pregnancies, regardless of risk other risk factors.

Accordingly, there is a need for an improved system and method of predicting preterm birth risk that overcomes inadequacies in the prior art.

### SUMMARY OF THE INVENTION

Generally speaking, in accordance with the invention a system and method are provided for identifying preterm birth risks by measuring cervical stiffness in pregnant patients, generating preterm birth risk factors and displaying warnings. Systems and methods in accordance with the invention can quantitatively measure the cervical tissue stiffness in pregnant people as a predictor for preterm delivery risk. Preferred designs include a cinching or cuffing mechanism that will wrap around the cervical tissue and compress the cervix with added air or liquid pressure, or with the cinching of a loop, squeezing the cervical tissue to a predetermined displacement. Preferred compressions are at least about 1, 2 or 3 mm, preferably about 3-4 mm of the diameter of the cervix. Expanding devices that measure the force needed for the expansion of cervical tissue are also disclosed. Obtaining accurate quantitative measurements from systems and methods in accordance with the invention is important, because of the ability of these systems to distinguish between a healthy and a too soft cervix as an indicator of preterm birth.

Systems and methods in accordance with the invention address an important concern of the top stakeholders, including pregnant people and OB/GYNs, that the device can accurately indicate and or display a risk of preterm birth by being able to measure the stiffness of the cervix and generate a cervical stiffness value score.

The measured elastic modulus from systems and methods in accordance with the invention should be lower for the softer tissue representing the softer cervix and higher for the stiffer tissue representing the healthy cervix. These values will be significantly different and this compression or expansion technique will indicate the stiffness of the cervix for preterm birth risk assessment and warning.

Accordingly, it is an object of the invention to provide an improved system and method for measuring cervical stiffness.

Another object of the invention is to provide an improved system and method for identifying cervical insufficiency as an indicator for preterm birth risk.

Still other objects of the invention will in part be obvious and will, in part be apparent from the specification and drawings. The invention accordingly comprises the devices, systems and methods which will be exemplified in the structures and methods hereinafter described, and the scope of the invention will be indicated in the claims.

### BRIEF DESCRIPTION OF THE DRAWINGS

For a fuller understanding of the invention, reference is made to the following description, in connection with the accompanying drawings, in which:

FIG. 1A is a graph showing the relationship between cervical length and preterm birth;

FIG. 1B is a graph showing the relationship between a cervical stiffness (softness) index and preterm birth;

FIGS. 2A and 2B show a side view of a piston-type probe for measuring cervical stiffness, in accordance with an embodiment of the invention;

FIGS. 3A and 3B show side views of an expansion-type probe for measuring cervical stiffness, in accordance with an embodiment of the invention;

FIGS. 4A, 4B, and 4C show a perspective view of a compression-type probe for measuring cervical stiffness, in accordance with an embodiment of the invention;

FIG. 5A is a schematic view of a string compression-type system for measuring cervical stiffness, in accordance with an embodiment of the invention;

FIG. 5B is a perspective view of the probe of the system shown in FIG. 5A;

FIG. 6A is an alternative perspective view of the probe for use with the system of

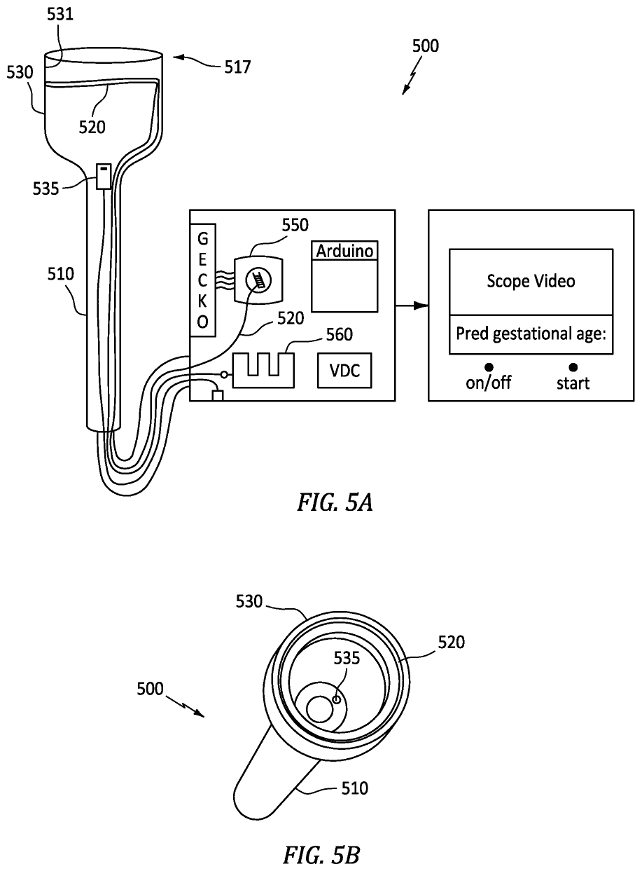

FIG. 5A; and

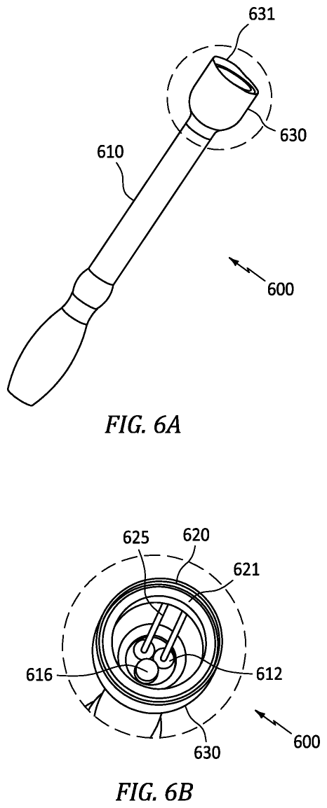

FIG. 6B is an enlarged view of the distal end of the probe of FIG. 6A.

The drawings are presented for purposes of illustration only. They are not necessarily to scale, and are not to be interpreted as limiting.

### DETAILED DESCRIPTION OF THE PREFERRED EMBODIMENTS

The invention relates to a system and method for measuring cervical stiffness in pregnant patients, to be used as an effective identifier of preterm birth risk.

During pregnancy, the cervix remodels to prepare for vaginal delivery. This remodeling includes a softening and shortening of the cervix. Cervical length, generally measured using transvaginal ultrasound, has been used as the clinical standard for predicting a risk of preterm birth. However, it has been determined that premature cervical softening is an accurate indicator of cervical insufficiency and that measuring cervical softening can be a more consistent indicator of preterm birth risk.

The invention provides a system and method to measure cervical stiffness, and therefore changes in cervical stiffness, using simple biomechanical measurements. Systems and methods in accordance with the invention provide more patients with the ability to monitor their risk of preterm birth more confidently and affordably. Accordingly, methods in accordance with the invention can be performed on patients that have not exhibited risk factors, as part of a normal office visit.

Preferred embodiments of the invention comprise an inflatable tube or string loop that surrounds the base of the cervix and compresses or cinches e.g., at least about 3 mm, preferably about 4 mm in diameter to compress the cervix. The string loop can be formed of a wire, polymer or natural fiber string and the like. The force required to compress the cervix is measured by measuring the resultant tension on the string at the point of the predetermined compression. The stiffness of the cervix is measured by identifying the force/tension needed to achieve the preselected deformation. The invention can also comprise a video scope, encased in the handle, that allows for speculum-free placement for optimal patient comfort. Stiffness can alternatively be measured by compressing the cervix with an expanding plug, similar in appearance to a tampon.

Parra-Saavedra, M., supra, describes how to calculate a cervical consistency index (CCI) percentage using ultrasound measurements. The anteroposterior cervical diameter is measured both before (AP) and after (AP′) application of manual pressure on the cervix. The CCI index is calculated using the formula: CCI=((AP′/AP)×100). Thus, a higher CCI value corresponds to a stiffer cervix.

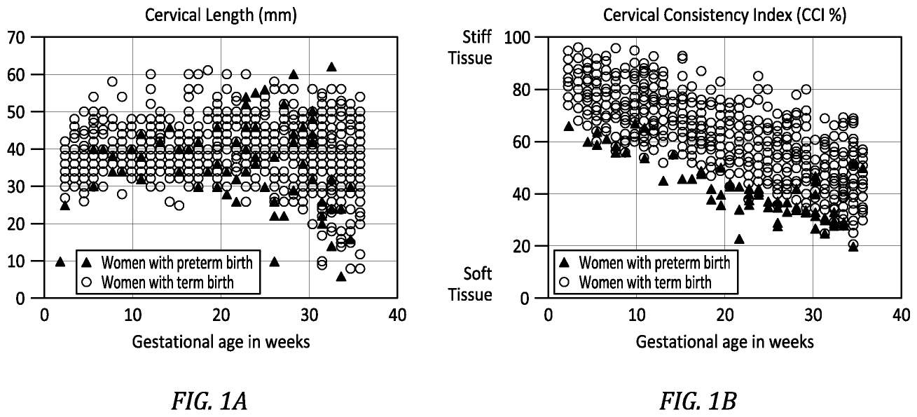

FIG. 1B, shows plots of CCI measurements according to gestational age at delivery (i.e. at term (>37 weeks) and preterm (≤37 weeks)). Multivariable logistic regression analysis of CCI measurements showed statistical significance in the prediction of preterm birth risk. Accordingly, an accessible device and method to quantify relative cervical stiffness over the course of a woman's pregnancy, in order to help determine cervical insufficiency and diagnose whether they are at high risk for preterm delivery based on cervical insufficiency is provided.

During pregnancy, the cervix remodels to prepare for vaginal delivery. This remodeling includes a softening and shortening of the cervix over the normal 39 week period. It is common for physicians to measure cervical length using transvaginal ultrasound to monitor high risk pregnancies and evaluate preparedness for delivery. Although cervical length has been considered the gold standard for detecting pregnancies at high risk for preterm birth, a comparison of FIGS. 1A and 1B shows that cervical consistency index (CCI), which involves cervical stiffness, should be a more reliable indicator for spontaneous preterm birth.

The CCI measurement is performed with ultrasound and is not widely performed clinically. Additionally, the technique for performing the measurement involves multiple steps and a highly trained ultrasound technician. The invention provides an alternative device and method for measuring cervical stiffness, which can be used to calculate a cervical stiffness score that relies on biomechanical measurements instead of ultrasonography. Systems and methods in accordance with the invention can be used during regular check-ups with the patient's obstetrician to monitor cervical stiffness beginning at the start of pregnancy.

For this measurement, there are a few considerations that will be addressed herein. First, systems and methods in accordance with the invention should account for the different anatomy present in different patients. Thus, it can be helpful to establish personal baseline measurements early in the pregnancy, so that changes in anatomy can be monitored as the pregnancy progresses. Devices in accordance with the invention should also be constructed and handled to minimize the possibility of introducing infections to patients. Additionally, the device should be sufficiently delicate to help ensure there is essentially no risk of interfering with the pregnancy or disrupting the structures that protect the internal fetus.

With a simple to use, non-painful device, cervical stiffness measurements can become widely accessible to pregnant patients and physicians to monitor cervical development over the gestational term and assess a risk for preterm birth. Therefore, the invention provides a convenient way to monitor cervical stiffness and development throughout pregnancy to help identify pregnancies at high risk for preterm birth.

As used herein, Preterm Birth (PTB) will refer to delivery before 37 weeks of gestation (or fewer than 259 days from first day of last menstrual cycle). 10% of all births are impacted by PTB as of 2020 (15 million babies). The cervix changes throughout pregnancy. It shortens in length and softens in stiffness. However, excessive softness too early in the pregnancy term can flag a preterm birth risk. Therefore, monitoring during the course of the pregnancy can be important.

Systems and methods in accordance with the invention should eliminate the need for a speculum; be usable with patients with different anatomies; not harm the baby (fetus) or patient; be comfortable; be easily sterilized for reuse; avoid infection; look approachable/unintimidating; avoid incorrect measurements caused by patient movement or contact force from practitioner. In one embodiment of the invention, the outer surface of devices in accordance with the invention are covered with medical grade silicone, to promote smooth placement and comfort to the patient.

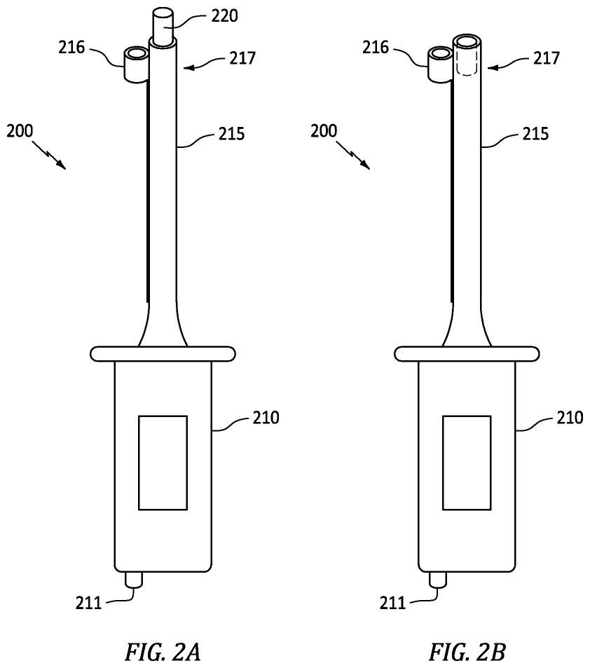

A first embodiment of a device for pressure-based measurement cervical stiffness in pregnant patients is shown generally in FIGS. 2A and 2B as a piston probe 200. Piston probe 200 includes a handle 210 and a small piston 220 extending from a distal end 217 of a neck 215 extending from handle 210. A scope 216 is positioned at distal end 217 to help with proper placement of piston 220, without the need for a speculum. An alternate scope design could position the scope at the distal end of piston 220, for a smoother profile. A jack 211 for connecting handle 210 to an electronic device (not shown) for powering probe 200 and interpreting the measurements detected with probe 200 is located at a proximal end of handle 210.

In one embodiment of the invention, distal end 217 of neck 215 is positioned against the cervix of a patient. The distal end of piston 220 is pushed against the cervix until a preselected amount of cervical deformation is detected. This amount can range from over about 2 to about 3-4 mm, depending on the personal anatomy of the patient and the initial cervical stiffness at the beginning of the pregnancy. This deformation can be measured with a distance sensor at the distal end of probe 200. The pressures needed to compress piston 220 before that preselected amount of cervical deformation is detected is measured with a pressure sensor associated with probe 200. Alternatively, piston 220 can be inserted in the retracted configuration and the pressure can be measured to identify the force needed to extend piston 220 the predetermined distance. In either embodiment, piston 220 is used to measure resistance to deformation (cervical stiffness). This value of cervical stiffness can be used to calculate a cervical stiffness score and help predict preterm birth risk. Thus, once the pressure needed for a preselected amount of cervical deformation is determined, piston probe 200 can display a preterm birth risk factor after comparing current cervical stiffness to historical stiffness measurements, taking gestational timing into consideration.

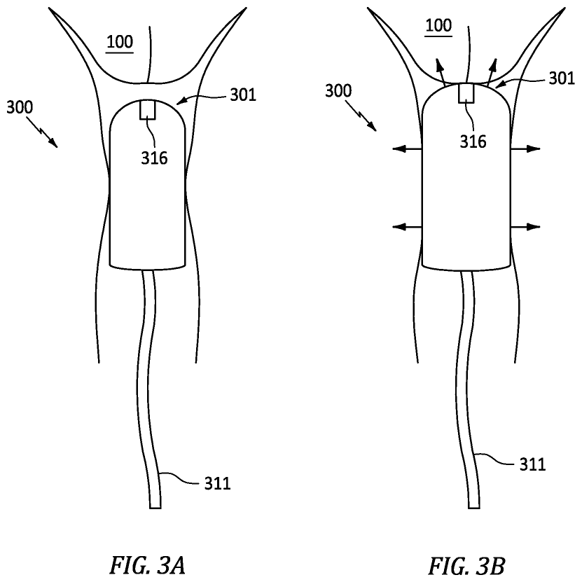

Another preferred embodiment of the invention is shown generally as a tampon-inspired expansion-type pressure detection probe 300, shown generally in FIGS. 3A and 3B. Tampon-style device 300 is inserted into the vagina, until a distal end 301 engages the patient's cervix 100. A scope 316 at distal end 301 can help with proper placement. A suctioning/gripping feature along the sides of device 300 can help maintain device 300 in proper position while the cervical stiffness measurement is performed. Tampon device 300 then expands in the distal direction for a predetermined distance against cervix 100. A pressure sensor at distal end 301 will be positioned against cervix 100 and measures the pressure exerted against by cervix 100 against device 300 from said preselected deformation. A transmission wire 311 is connected to electronics for interpreting the force needed to deform cervix 100 the predetermined amount. The stiffness score can be calculated and preterm birth risk can be assessed and/or displayed, based on these and earlier measurements.

Another preferred embodiment of the invention can be referred to as the cuff design. Such device should have a “cuff” sized and configured to surround the cervix and impose a circumferential deformation with the application of compressive force. Three design iterations of the cuff design are discussed below.

A first embodiment of the cuff design employs an air pump and pressure sensor to inflate the cuff around the cervix, similar to a blood pressure cuff. With this first embodiment, the cuff is inflated and the pressure measurement correlates to stiffness of the cervix, as a stiff cervix will require greater pressure to compress the cuff the pre-determined amount. Thus, as the inner circumference of the cuff decreases the diameter of the cervix will be compressed.

Compressions can vary with individual cervix geometries. However, in general, cervical diameter compressions of over 1 or 2 mm, preferably about 1-5 mm, more preferably about 3-4 mm are effective to measure cervical stiffness. A second embodiment of the cuff design uses hydraulics and a liquid pressure sensor. A third embodiment of the cuff design employs a tension string and measures the tension on the string as the selected amount of compression is achieved. The string can be wire, polymer, natural fiber or the like.

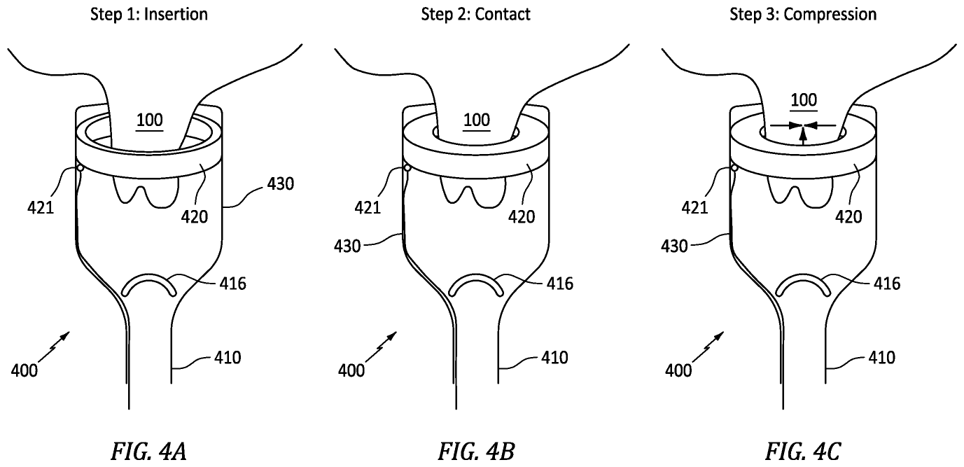

An inflatable (air or liquid) cuff design device in accordance with a preferred embodiment of the invention is shown generally as a balloon ring probe 400 in FIGS. 4A to 4C. Balloon ring probe 400 is formed with a handle 410, designed to hold a miniaturized syringe pump and tubing system operatively coupled in fluid communication with an inflatable balloon ring 420. Balloon ring probe 400 preferably has a plastic exterior and can be covered with medical grade silicone. Inflatable balloon ring 420 is located on the inside surface of a head 430 of probe 400. A scope 416 is located in head 430 to help with proper placement. Sensors, such as sensor 421 detect displacement of balloon 420.

As shown in FIG. 4A, balloon ring 420 is placed over a patient's cervix 100. Next, balloon 420 is inflated until it contacts the cervix. Probe 400 can be calibrated to detect how much pressure is needed to inflate balloon ring 420. Therefore, once that threshold is met, balloon ring probe 400 will recognize that engagement with cervix 100 is achieved. For example, a spike in pressure can signal engagement with the cervix and initiation of cervical compression. Probe 400 is constructed and configured to measure how much balloon 420 has been inflated and therefore, it will identify the initial cervix diameter. Then, balloon 420 is inflated and compresses cervix 100, until probe 400 measures a pre-selected amount of cervical compression (preferably over about 1 or 2 mm, more preferably about 1-5 mm, even more preferably about 3-4 mm). Once this amount of cervical compression is measured, probe 400 will measure how much pressure was needed to compress the cervix the pre-selected amount. Alternatively, once contact is detected, the device can measure the amount of compression diameter for a pre-selected pressure of compression. In either event, the cervical stiffness score can then be calculated and a pre-term birth risk factor score can be displayed.

A cinch-string probe 500 iteration of the cuff design, in accordance with another preferred embodiment of the invention, is shown generally in FIGS. 5A and 5B. Cinch-string probe 500 includes a handle 510 having a head 530 at a distal end 517 thereof. Head 530 includes a flexible inelastic string 520 formed of wire, polymer or natural fibers around an inner surface 531 of head 530. A scope 535 can be present within head 530, to help with proper placement of head 530. Once string 520 is located around the cervix of the patient, string 520 is tightened by shortening the length of the loop, such as with a stepper motor 550. A tension sensor 560 is used to measure the tension on string 520 and will be able to detect initial engagement, such as with a spike in tension.

As stepper motor 550 tightens string 520 around the cervix, tension sensor 560 will detect once close contact is made as string 520 tightens around a patient's cervix. Stepper motor 550 will continue to tighten string 520 around the cervix, until a preselected length of string, corresponding to a preselected compression of the diameter of the cervix, is detected. For most patients, this will be over 1-2 mm or about 1 to 5 mm, preferably about 3 to about 4 mm, most preferably about 3.5 mm. Pressure sensor 560 will detect the amount of tension needed to achieve this preselected compression. From this measurement and earlier measurements, the change in stiffness of the patient's cervix and the patient's cervical stiffness can be calculated. If the stiffness exceeds a predetermined threshold cervical stiffness for the appropriate gestational age, there is an indication of a danger of a preterm birth. A warning/risk factor can be displayed.

A cinch-string probe 600 in accordance with another embodiment of the invention is shown generally in FIGS. 6A and 6B. Cinch-string probe 600 is similar to probe 500, and includes a handle 610, a head 630, a scope 616, and a flexible string loop 620. A pair of large tubes 612 guide string 620 down handle 610. A pair of small, rigid guide tubes 625 help keep string 620 in proper position and help prevent string 620 from becoming tangled. A thin pliable membrane 621 can be positioned between string 620 and the cervix for comfort and protection, as well as to keep string 620 in the proper location prior to cinching. Head 630 (and the other probes disclosed herein) can be formed with a flexible lip 631 to assist with comfortable insertion into the patient's vagina to engage the cervix. Throughout cinching, a user can monitor the tension of string 620 with e.g., a load cell. The data can be used to generate load displacement curves. The liner regions can be fit to extract the slopes from which cervical stiffness is calculated, a stiffness score determined, and when appropriate, a risk factor or warning displayed.

It will thus be seen that the objects set forth above, among those made apparent from the preceding description, are efficiently attained, and, since certain changes may be made in carrying out the above method and in the devices and compositions set forth without departing from the spirit and scope of the invention, it is intended that all matter contained in the above description and shown in the accompanying drawings shall be interpreted as illustrative and not in a limiting sense.

It is also to be understood that the following claims are intended to cover all of the generic and specific features of the invention herein described and all statements of the scope of the invention which, as a matter of language, might be said to fall therebetween.

What is claimed is:

1. A method for determining cervical stiffness in a patient having a cervix, comprising:
inserting a probe to a position wherein a distal end of the probe engages the cervix;
activating the probe to deform the cervix a predetermined first amount;
identifying the amount of force needed for the probe to deform the cervix the first amount; and
identifying an initial cervical stiffness value based on how much force was needed to deform the cervix the first amount.

2. The method of claim 1, wherein the cervix is deformed by compressing the cervix the first amount.

3. The method of claim 1, wherein the cervix is deformed the first amount by placing a ring-shaped cuff around the cervix and reducing an inner diameter of the cuff to compress the cervix the first amount.

4. The method of claim 3, wherein the cuff comprises an inflatable tube and the inner diameter of the tube is reduced by inflating the tube with a gas.

5. The method of claim 3, wherein the inflatable tube is inflated with a liquid.

6. The method of claim 3, wherein the force needed to compress the cervix the first amount is measured by measuring the pressure needed to inflate the cuff enough to reduce the inner diameter enough to compress the cervix the first amount.

7. The method of claim 3, wherein the cuff comprises a string, and the inner diameter is reduced to compress the cervix the first amount by shortening a length of the string around the cervix.

8. The method of claim 7, wherein the force needed to compress the cervix the first amount is measured by measuring a tension on the string after it compresses the cervix the first amount.

9. The method of claim 1, wherein the first amount is a cervical compression in a diameter of the cervix of at least about 2 mm.

10. The method of claim 1, wherein the first amount is a cervical compression in a diameter of the cervix of about 1-5 mm.

11. The method of claim 1, wherein the probe includes a piston at the distal end of the probe, the cervix is deformed the first amount by pressing the piston into the cervix, and the force needed to deform the cervix the first amount is measured by measuring the force on the piston when it deforms the cervix the first amount.

12. The method of claim 1, wherein the patient has a length of gestational term and the amount of force needed to deform the cervix the first amount is below a predetermined threshold value associated with the patient's gestational term, the patient is designated to be at a preterm birth risk.

13. The method of claim 3, wherein the patient has a length of gestational term and the amount of force needed to compress the cervix the first amount is below a predetermined threshold value associated with the patient's gestational term, a preterm birth risk warning is displayed.

14. A device for measuring cervical stiffness, comprising
a probe constructed and configured to be inserted into a patient's vagina, having a proximal end and a distal end;
a head located on the distal end, the head having an inner surface sized, configured and adapted to engage a cervix of the patient;
a compression cuff located at the inner surface, the cuff adapted, sized, positioned and configured to surround the patient's cervix, the compression cuff functionally coupled to a pressure sensor;
an activator adapted to trigger the compression of the cuff, and the compression cuff and pressure sensor adapted, configured and constructed to measure and identify the force required to compress the inner diameter of the cuff a first amount around the patient's cervix.

15. The device for measuring cervical stiffness of claim 14, comprising a distance sensor adapted to measure the first amount the cervix is compressed by the cuff.

16. The device for measuring cervical stiffness of claim 14, comprising a force sensor adapted to measure the amount of force needed for the cuff to compress the cervix the first amount.

17. The device for measuring cervical stiffness of claim 14, wherein the cuff is an inflatable tube adapted to be inflated with gas or liquid.

18. The device for measuring cervical stiffness of claim 14, wherein the cuff is a flexible loop, adapted to compress the cervix when a length of the loop is shortened.

19. The device for measuring cervical stiffness of claim 14, and comprising a video scope on the distal end.

20. The device for measuring cervical stiffness of claim 16, wherein the loop is formed from flexible wire, polymer or natural fiber material.
